(19) United States
(12) Patent Application Publication
(10) Pub. No.: US 2026/0207078 A1
(43) Pub. Date: Jul. 23, 2026

(54) PLANTAR PRESSURE MEASUREMENT SYSTEM AND METHOD BASED ON AIR PRESSURE SENSOR AND AIRBAG

(71) Applicant: iFuturelab Group Holding Limited, TORTOLA (VG)

(72) Inventor: Daniel BROOKS, TORTOLA (VG)

(21) Appl. No.: 19/026,901

(22) Filed: Jan. 17, 2025

(51) Int. Cl. A61B 5/103; A61B 5/00

(52) U.S. Cl. CPC A61B 5/1038; A61B 5/0022; A61B 5/486; A61B 5/6807; A61B 5/7267; A61B 5/7275; A61B 5/7282; A61B 2562/0247; A61B 2562/168

(57) ABSTRACT

The present application discloses a plantar pressure measurement system and method based on air pressure sensor and airbag, the system is embedded in a shoe sole, including: an adjustment module for sensing the plantar pressure, obtaining the pressure data, and adjusting the air pressure inside each of the airbags in accordance with an adjustment instruction; a data processing module for receiving and pre-processing the pressure data to obtain the plantar pressure information, and generating a plantar pressure health report as well as the adjustment instruction wireless communication module for transmitting the plantar pressure health report to an external device or the cloud, and transmitting user feedback to the data processing module; real-time feedback module for providing abnormal pressure state feedback to the user; and power management module for providing electric power, and real-time monitoring of the power state of the system.

### TECHNICAL FIELD

The present disclosure relates to a technical field of plantar pressure detection, and particularly to a plantar pressure measurement system and method based on air pressure sensor and airbag.

### BACKGROUND

In the field of plantar health monitoring, plantar pressure detection technology has been widely used in gait analysis, sports monitoring, and rehabilitation training scenarios, etc. Accurate detection of plantar pressure distribution not only helps to prevent foot diseases, but also improves gait, enhances athletic performance, and provides important data support in rehabilitation therapy. Traditional plantar pressure measurement systems usually rely on rigid sensors, which have many limitations in practical applications. For example, the rigid material properties of rigid sensors determine that they can affect wearing comfort and even normal gait, especially for the elderly, chronically ill, or users in rehabilitation, and these sensors do not have sufficient measurement accuracy in the face of complex and changeable gait and terrain.

In addition, the plantar pressure measurement system in the prior art usually lacks a real-time feedback and early warning mechanism, so that when the user's plantar pressure distribution is abnormal, it cannot provide timely alerts at the early stage of the abnormality, thus delaying the best time for early intervention, and this lagging monitoring method obviously fails to satisfy the growing demand for health management.

In view of this, there is an urgent need to provide a high precision, real-time and comfortable plantar pressure monitoring program.

### SUMMARY

Based on the above application requirements and technical background, in order to solve the technical problem of the prior art that plantar pressure monitoring cannot be performed simultaneously in real time, comfortably and with high precision, the present application adopts the following technical solution:

A first aspect of the present application proposes a plantar pressure measurement system based on air pressure sensor and airbag, the system is embedded in the sole of a shoe, comprising an adjustment module, a data processing module, a wireless communication module, a real-time feedback module and a power management module;

the adjustment module comprises a plurality of independently working airbags, a plurality of air pressure sensors, an air pump and a plurality of air valves for sensing the plantar pressure, obtaining the pressure data and transmitting the same synchronously to the data processing module, and adjusting the air pressure inside each of the airbags in accordance with an adjustment instruction;

the data processing module is connected to the air pressure sensor and comprises a pre-processing unit and an algorithm unit for receiving, pre-processing the pressure data to obtain plantar pressure information, generating a plantar pressure health report and the adjustment instruction;

the wireless communication module for transmitting real-time the plantar pressure information and the plantar pressure health report to an external device or a cloud, and transmitting user feedback inputted by a user from the external device or the cloud to the data processing module;

the real-time feedback module for providing abnormal pressure state feedback to the user when an abnormal pressure state is detected, the abnormal pressure state feedback being provided in a manner comprising vibration, sound or visual feedback;

the power management module for providing power, real-time monitoring the power status of the system, and controlling the power consumption of the plantar pressure measurement system.

Further, each of the airbags corresponds to a key pressure region embedded in the sole, the key pressure region corresponding to a key pressure point on the sole of the foot, comprising a heel region, a front metatarsal region, a lateral foot region, and a metatarsal arch region.

Further, each of the airbags corresponds to one or more of the air pressure sensors; the adjustment module further comprises a weigh sensor; each time the plantar pressure information, plantar pressure health report, and the adjustment instruction obtained by the processing of the data processing module are stored in the system's memory or in the cloud as a user history data.

Further, the algorithm unit comprises a first preset algorithm for generating a plantar pressure distribution map and a plantar pressure change trend, calculating a plurality of key gait indicators, and extracting a plurality of gait characteristics; the gait indicators comprise a center of pressure, a center of gravity transfer trajectory, as well as a plantar touching area and touching time; the gait characteristics comprise a step speed, a step speed, a plantar landing mode, and a gait symmetry.

Further, the algorithm unit further comprises a second preset algorithm for identifying an abnormal pressure state;

the second preset algorithm is a machine learning algorithm;

the abnormal pressure state comprises center of gravity shift, abnormal pressure distribution, gait instability, excessive internal and external rotation, or gait asymmetry, and the second preset algorithm recognizes the plantar pressure information exceeding the abnormal pressure threshold as the abnormal pressure state;

the abnormal pressure threshold is adaptively and dynamically adjusted based on the user history data and the plantar pressure information.

Further, the algorithm unit further comprises a third preset algorithm by which a personalized plantar pressure health report is generated based on the user history data, the results of the first preset algorithm, and the results of the second preset algorithm, and the plantar pressure health report is transmitted to the external device through the wireless communication module;

the plantar pressure health report comprises the abnormal pressure state, the plantar pressure distribution map and plantar pressure change trend, the gait indicators and gait characteristics, a personalized health advice, and a long-term health trend analysis report.

Further, the algorithm unit further comprises a fourth preset algorithm for generating the adjustment instruction and transmitting it to the adjustment module for adjusting the air pressure of the airbag based on the adjustment instruction through the air pump and the plurality of air valves.

Further, the power management module adopts a low power consumption design and comprises a battery pack, a power monitoring circuit and an intelligent power controller.

A second aspect of the present application proposes a plantar pressure measurement method based on air pressure sensor and airbag, the method comprising:

sensing the plantar pressure, obtaining the pressure data and transmitting the same synchronously to the data processing module, and adjusting the air pressure inside each of the airbags in accordance with the adjustment instruction through an adjustment module, the adjustment module comprises a plurality of independently working airbags, a plurality of air pressure sensors, an air pump and a plurality of air valves;

receiving, pre-processing the pressure data to obtain plantar pressure information, generating a plantar pressure health report and the adjustment instruction through a data processing module, the data processing module is connected to the air pressure sensor and comprises a pre-processing unit and an algorithm unit;

transmitting real-time the plantar pressure information and the plantar pressure health report to an external device or a cloud, and transmitting user feedback inputted by a user from the external device or the cloud to the data processing module through a wireless communication module;

providing abnormal pressure state feedback to the user when an abnormal pressure state is detected through a real-time feedback module, the abnormal pressure state feedback being provided in a manner comprising vibration, sound or visual feedback;

providing power, real-time monitoring the power status of the system, and controlling the power consumption of the plantar pressure measurement system through a power management module.

Further, each of the airbags corresponds to a key pressure region embedded in the sole, the key pressure region corresponding to a key pressure point on the sole of the foot, comprising a heel region, a front metatarsal region, a lateral foot region, and a metatarsal arch region.

Compared with the prior art, the beneficial effects of the present application are:

The plantar pressure measurement system based on air pressure sensor and airbag and the method provided by the present application, by embedding a flexible airbag in the sole of the shoe, ensures the adaptability and comfort of the system to the foot in the process of using the shoe, especially in the case of prolonged use, avoids the problem of localized pressure concentration that may be caused by the rigid sensor, and enhances the user's comfort of wearing the shoe; and senses the change of the air pressure inside the airbag through the air pressure sensor to realize the high-precision monitoring of plantar pressure through the air pressure sensor, more effective response to complex terrain and gait changes, to provide more stable and reliable measurement data, through the combination of air pressure sensors and airbags, the system is able to provide higher precision and sensitivity than the traditional rigid pressure sensors, especially in the complex gait conditions, but still able to maintain a stable measurement performance; through the real-time monitoring of the distribution and changes in the plantar pressure and based on the real-time data Through real-time monitoring of plantar pressure distribution and changes and algorithmic analysis based on real-time data, the system generates plantar pressure distribution maps, identifies gait characteristics and abnormal plantar pressure states, and based on the user's own needs, provides instant feedback and early warning functions (such as vibration, sound or visual cues) and personalized health advice and rehabilitation guidance, which can meet the user's needs for real-time monitoring of plantar health and personalized dynamic management, thus improving the performance of the plantar pressure measurement system and the user's experience. The system not only provides real-time plantar pressure monitoring, but also generates personalized analysis reports and health recommendations based on the user's gait characteristics, which is of practical significance for athletes, rehabilitation patients, and daily health management users.

The plantar pressure measurement system based on air pressure sensor and airbag provided in the present application can be used in the field of rehabilitation training to assist rehabilitation therapy by real-time monitoring of gait changes of patients, and can be applied to the gait monitoring of rehabilitation patients, and can be used in the real-time tracking of pressure changes and gait characteristics to help the rehabilitation therapist to assess the patient's recovery progress and provide personalized training programs; and can be used in the field of sports monitoring to Help athletes optimize their gait and reduce the risk of sports injuries. For athletes, the system can provide detailed gait analysis reports to help them optimize their running or walking postures and avoid sports injuries due to poor gait; it can also be used in the field of health assessment to detect gait anomalies, plantar pressure concentration problems, etc. By analyzing gait anomalies, it can detect early health problems, such as unstable gait in the elderly, It can also be used in the field of health assessment to detect gait abnormalities and detect early health problems such as unstable gait in the elderly and foot ulcer risk in diabetic patients.

### BRIEF DESCRIPTION OF THE DRAWINGS

In order to more clearly explain the technical solutions in the present disclosure or the prior art, drawings required in the embodiments or the prior art will be briefly described below. Obviously, the drawings in the following description are some embodiments of the present disclosure. For those skilled in the art, other drawings may be obtained from these drawings without any creative effort.

FIG. 1 shows a schematic diagram of a plantar pressure measurement system based on air pressure sensor and airbag provided by the present application;

FIG. 2 is a schematic diagram of a flow block diagram of a plantar pressure measurement method based on air pressure sensor and airbag provided by the present application.

### DETAILED DESCRIPTION OF THE EMBODIMENTS

The present application proposes a plantar pressure measurement system based on air pressure sensor and airbag, and in order to describe the present application more specifically, the technical solutions of the present application are described in detail below in connection with the accompanying drawings and specific embodiments, and it should be understood that the specific embodiments described herein are only for explaining the present application, and are not intended to limit the present application. Based on the embodiments in this application, all other embodiments obtained by a person of ordinary skill in the art without making creative labor fall within the scope of protection of this application.

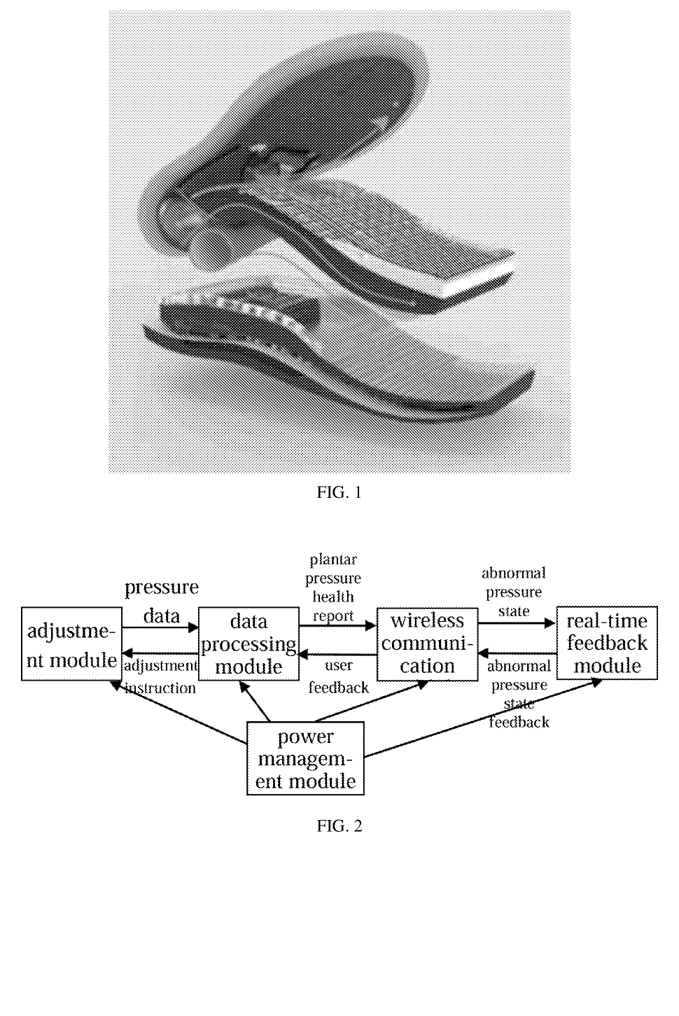

A first aspect of the present application proposes a plantar pressure measurement system based on air pressure sensor and airbag, the system is embedded in the sole of a shoe, as shown schematically in FIG. 1, and specifically comprising: an adjustment module, a data processing module, a wireless communication module, a real-time feedback module and a power management module;

The adjustment module comprises a plurality of independently working airbags, a plurality of air pressure sensors, an air pump and a plurality of air valves, wherein the airbags are used for sensing the pressure of the sole of the foot, wherein the air pressure sensors are used for detecting the air pressure inside the airbags, obtaining the pressure data and synchronizing the same for transmitting the same to the data processing module, and wherein the air pump and the plurality of air valves are used for adjusting the air pressure inside the respective the airbags according to the adjustment instructions;

Wherein the airbag is made of a flexible and highly elastic material, including a silicone or polyurethane material, the flexible and highly elastic material is highly elastic, wear-resistant and lightweight, and is not only able to withstand multiple repetitions of pressure action, but also deforms in accordance with the shape of the user's sole of the foot and changes in the plantar pressure applied to it, so that it is able to better adapt to the shape and movement of the sole of the foot, and ensures that pressure applied to different areas of the sole of the foot can be accurately sensed through the deformation of the airbag. This ensures that the pressure exerted on different areas of the foot can be accurately sensed through the deformation of the airbag, while maintaining comfort and durability over long periods of wear. Compared to a traditional rigid pressure sensor, the airbag of the flexible structure conducts pressure through a gas, is softer and fits the foot better, enables more uniform distribution and sensing of pressure changes on the sole of the foot, and reduces interference with the user's gait.

In one embodiment, each of the airbags corresponds to a key pressure region embedded in the sole, and the key pressure region corresponds to a key pressure point of the sole of the foot, including a heel region, a front metatarsal region, a lateral foot region, and a metatarsal arch region.

Wherein the heel region is used to detect pressure changes in the heel, the heel is the region of maximum pressure in gait, bearing the main vertical load of the human body in standing and walking, especially at the moment of landing, the pressure is concentrated and the impact force is large, so the airbag set in the heel region is larger and thicker, with high cushioning and shock-absorbing properties, in order to lessen the impact of the heel and protect the joints and the spine of the foot, and to accurately monitor pressure changes in this region;

The front metatarsal region is used to detect pressure changes in the forefoot, and the front metatarsal provides thrust output when the user walks or runs, and the force changes are dynamic and complex, requiring both support and flexibility, so the airbag set in the front metatarsal region is thinner, designed to be flat or strip-shaped, and set to be more flexible, so as to provide accurate pressure sensing and moderate support;

The lateral foot region is used to detect pressure changes on the lateral side of the foot, the lateral side of the foot mainly bears dynamic loads during walking, especially in the transition phase of the foot when the foot exerts force from the heel to the forefoot, and by analyzing the force on the lateral foot region it can help to analyze the balance of the user's gait and whether there is any problem with the foot's outward or inward roll, and the airbag set in the lateral foot region is designed as an elongated shape covering the area of the outer edge of the sole, which is consistent with the direction of the plantar force line, and is set in a flat or strip shape, thus providing precise pressure sensing and moderate support. consistent with the direction of the line of force of the sole of the foot, thereby providing stability and support to avoid lateral roll-over or sprain;

The metatarsal arch region is used to detect pressure changes in the arch of the foot, and the metatarsal arch (also known as the arch of the foot) is an important cushioning structure of the sole of the foot, which can help to disperse pressure and maintain the stability of the foot, and by analyzing the force in the metatarsal arch region it can reflect the health status of the arch of the foot, which is important for evaluating the abnormality of the structure of the foot or the damage, especially for the users of flat feet or high arch of the foot, and the airbag set in the metatarsal arch region is designed to be in an arch shape that matching the shape of the arch and covering the key support area of the metatarsal arch, thereby preventing the arch from collapsing due to prolonged loading.

Further, the key pressure region also includes a toe region for detecting pressure changes in the toes, which are less pressurized in gait and play an auxiliary supporting and balancing role in propulsive movements, and the airbag set in the toe region is a slender and flexible small structure matching the shape of the toes, and the airbag has a thin structure emphasizing a high sensitivity to capture the subtle pressure changes in the toe region.

Therein, the size, thickness and shape of the airbag are adjustable, and can be adjusted according to the key pressure region and the actual application scenario in which it is set, so as to effectively balance the relationship between detection accuracy, functionality and user experience. For example, in sports or rehabilitation training scenarios, the soles of the feet are subject to higher impact and dynamic loads, and precise pressure detection is also required, the airbags are designed to be thicker in order to hold more gases, distribute the pressure on the soles of the feet more evenly, and provide a more comprehensive pressure sensing and a better cushioning and shock absorbing effect; in daily wear scenarios, the user pays more attention to lightweight, comfort, and the appearance of the shoes, and the demand is mainly focusing on basic health monitoring, the airbag is designed to be thinner and lighter to ensure the comfort and aesthetic appearance of the shoes.

In practical application, the present application combines the shape and mechanical characteristics of the sole of the foot with the arrangement of the airbag, which can flexibly adapt to the sole structure of different users, and when the user walks or stands, the pressure exerted by different areas of the sole of the foot leads to changes in the air pressure inside the airbag, so that the airbag can accurately reflect the corresponding changes in the pressure of the sole of the foot, which not only improves the accuracy of the perception of the pressure of the sole of the foot, but also improves the support and protective function of the shoes on the sole of the foot. support and protection function of the shoe.

In one embodiment, each airbag corresponds to one or more air pressure sensors for detecting changes in air pressure within the airbag, and converting the changes in air pressure into electrical signals, obtaining pressure data and synchronously transmitting the data to the data processing module for processing to ensure consistency of the data and temporal matching, so as to realize real-time monitoring of the pressure distribution of the various regions of the plantar foot. Specifically, when each airbag is independently connected to an air pressure sensor, a plantar pressure monitoring unit is formed for precise zonal monitoring, and when each airbag is connected to a plurality of air pressure sensors, multi-dimensional pressure data from the same region can be collected, and a plurality of air pressure sensors can be distributed in different directions and positions to enhance the coverage and sensitivity of monitoring and capture dynamic pressure fluctuations and directional changes, and even if a sensor fails, the redundancy design can also ensure that the pressure data are consistent and time-matched. Even if a sensor fails, the redundant design can ensure normal system function.

Wherein the air pressure sensor is connected to the airbag through a flexible air tube, thereby ensuring that changes in air pressure inside the airbag can be transmitted to the air pressure sensor in real time. The transfer of plantar pressure to the air pressure sensor through the air tube can absorb the deformation of the sole of the shoe, effectively avoiding direct stress on the air pressure sensor, and reducing damage to the sensor caused by direct extrusion or impact.

In practical application, whenever the user walks or stands, the pressure in different regions of the plantar foot will cause changes in the air pressure inside the airbag, and the air pressure sensor can capture these subtle changes in air pressure and convert these changes in air pressure into electrical signals (i.e., pressure data) outputs, and the strength of the electrical signals is directly proportional to the changes in air pressure, reflecting the pressure exerted by the various regions of the plantar foot, so as to provide an accurate gait analysis for subsequent data support.

Wherein the air pressure sensor has high sensitivity, high accuracy and high sampling frequency, and is capable of detecting minute air pressure changes within the airbag in real time, thereby accurately capturing minute changes in the plantar pressure of the user during walking or exercising, in particular minute pressure fluctuations of an athlete during fast running. In one embodiment, the air pressure sensor is miniaturized with a sampling frequency of up to several hundred hertz, ensuring that minute changes in each gait cycle can be captured without affecting wearing comfort.

In one embodiment, the material of the air pressure sensor is anti-fatigue and high temperature resistant, and has a moisture resistant coating design to ensure that high sensitivity can be maintained under repeated pressure (e.g., hundreds of pressures per minute during running) and complex environments (e.g., high temperatures, humidity, or strenuous exercise scenarios), to ensure continuous accuracy of the monitoring data, and to prevent damage due to external impacts or moisture intrusion.

In one embodiment, the air pump and the plurality of air valves are used to adjust the air pressure within the airbag according to an adjustment instruction, ensuring that the airbag is within a standard working air pressure range, avoiding measurement errors caused by too low or too high air pressure, and thus enhancing detection accuracy.

Specifically, air is compressed or inhaled through the air pump, and the flow of gas between the air pump and the airbag is regulated through the air valve to realize the inflation or deflation of the airbag, and the air pressure is dynamically regulated to ensure that each airbag is in an optimal working state.

In one embodiment, the adjustment module further comprises a weigh sensor, the pressure data further comprises weight data, the weigh sensor is located below the airbag for detecting the weight distribution and changes in various regions of the sole of the foot to obtain the weight data. The weigh sensor and the air pressure sensor complement each other, and the weight data, when combined with the output of the air pressure sensor, can provide the system with more comprehensive pressure analysis data, helping to more accurately analyze the pressure distribution of the various regions of the sole of the foot, and thus further realizing the accurate detection of the pressure distribution of the various regions of the sole of the foot.

Wherein the weigh sensor is robust and able to withstand the pressure of different users' body weights with high sensitivity and accuracy.

Further, the airbag, air pressure sensor, air pump and air valve are set to be removable, and the user carries out maintenance, replacement and function upgrading, for example, when the airbag leaks due to wear and tear, or when the performance of the air pump decreases, etc., the user can directly carry out disassembly and replacement, and by increasing the number of airbags and air tubes, the user can conveniently expand the multi-point monitoring capability.

The data processing module is connected to the air pressure sensor, comprising a pre-processing unit and an algorithm unit for receiving and pre-processing the pressure data to obtain plantar pressure information, generating a plantar pressure health report and the adjustment instructions;

Further, each time the plantar pressure information, plantar pressure health report, and the adjustment instructions obtained by processing of the data processing module are stored as the user history data in the memory of the system or in the cloud.

Wherein, the data processing module receives the pressure data in real time through a high-speed interface to ensure synchronized recording of pressure changes in all plantar regions. The high-speed interface ensures that the pressure data is transmitted to the data processing module in milliseconds time, avoiding delays and enabling the data to be received and analyzed in real time in response to the user's dynamic gait and pressure changes.

In one embodiment, the pre-processing unit is used to pre-process the received pressure data, the pre-processing comprising filtering, smoothing, denoising, and calibration.

Specifically, the preprocessing step of filtering is used for retaining useful frequency components in the data, removing unnecessary noise or interfering signals, ensuring the accuracy of the data, and includes: low-pass filtering, Kalman filtering, and adaptive filtering;

Wherein the low-pass filtering is used to pass low-frequency signals and eliminate high-frequency noise, this is because in plantar pressure detection, real pressure signals are mainly concentrated in the low-frequency band (such as changes in gait cycles), while high-frequency components are mostly noise or interference, and the threshold of the filter used for low-pass filtering can be adjusted according to the application scenario;

The Kalman filtering is used for dynamic noise removal in gait monitoring, and Kalman filtering is a recursive filtering method that can estimate the optimal solution of the current state by combining current data and historical data, and gradually approximates the real signal by dynamically adjusting the deviations between predicted values and actual observed values, and is applied to dynamic and complex scenes, and is especially suited to systems with a certain degree of randomness or process noise, for example, the user of the present application in the pressure signals that are constantly changing due to irregular movements;

The adaptive filtering is used to adjust filtering parameters in real time according to data characteristics, and the adaptive filter adapts to dynamic changes in signal and noise by adjusting the parameters of the filter in real time, and this filtering method does not need to pre-determine fixed filtering parameters but is dynamically adjusted according to the environment and data characteristics, and is suitable for complex or unpredictable environments, such as when the user walks on an uneven ground or quickly switches to different activity modes , ensuring that the system obtains clear signals in different gait or movement states.

Specifically, the pre-processing step of smoothing is used to further eliminate subtle fluctuations in the data and generate signals with better continuity by removing short-term random variations, ensuring that the data clearly reflects the gait characteristics of the user without being affected by short-term fluctuations, and comprises moving average and exponential smoothing;

wherein the moving average generates a smoother curve by calculating the average value of data points within a sliding window, which enables rapid reduction of data fluctuations and is suitable for static or low to medium frequency dynamic detection;

The exponential smoothing generates progressively changing signals by weighting historical data and with higher weighting of recent data, which is faster in response, enables real-time processing, and is applicable to scenarios with faster gait changes.

Specifically, the pre-processing step of denoising is used to further eliminate random noise in the data while retaining the true signal to ensure smooth and reliable data, including wavelet transform;

Wherein the wavelet transform decomposes the signal into subbands of different frequencies by wavelet decomposition, reconstructs the signal after removing the high frequency noise component, is applicable to non-smooth signals, retains more detailed features, and is capable of realizing a fine analysis of plantar pressure changes in dynamic detection.

Specifically, the calibration processing is used to adjust the raw signals output from the sensors to real physical quantity values to eliminate sensor bias and systematic errors, ensuring that each sensor can maintain accuracy under different temperature, humidity and pressure environments.

In one embodiment, the algorithm unit comprises a first preset algorithm for generating a plantar pressure distribution map and a plantar pressure change trend, calculating key gait indicators, and extracting gait characteristics;

First, the first preset algorithm summarizes the plantar pressure information of key pressure regions of the plantar foot at various time points, thereby generating the plantar pressure distribution map and the plantar pressure change trend. The plantar pressure distribution map and the plantar pressure change trend intuitively display, in a visualized form, the stress on different regions of the plantar foot and the change trend over time when the user is walking or standing, and by displaying the stress and change of the plantar pressure in a graphical form, it is possible to clearly display the pressure concentration region of the plantar foot, the pressure deviation, the pressure anomaly, the pressure symmetry of the regions, and the pressure change trend;

Specifically, by continuously collecting and analyzing the pressure data through the first preset algorithm, the plantar pressure distribution graph and the plantar pressure change trend are dynamically changing. And since the plantar pressure is also not static and unchanging, but dynamically changes with the user's pace and movement changes, the dynamic plantar pressure distribution map and the plantar pressure change trend can demonstrate how the pressure transfers between different regions of the plantar when the user is walking or running, and thus analyze the user's center of gravity changes and foot landing situation, and help identify the balance and stability of the gait;

Secondly, the first preset algorithm calculates gait indicators through the plantar pressure distribution map and plantar pressure change trend, the gait indicators including the center of pressure, the center of gravity transfer trajectory, as well as the plantar touching area and the touching time, which help analyze the gait health condition of the user;

Specifically, the center of gravity is obtained by dynamically calculating the location of the center of gravity of the pressure distribution, and the center of gravity is used to indicate the average force point of the sole of the foot, reflecting the gait stability and the distribution of the load of the sole of the foot; the center of gravity transfer trajectory is obtained by generating the trajectory and feature extraction, and the center of gravity transfer trajectory describes how the center of gravity of the user transfers from the heel of the foot to the front metatarsal during a gait cycle, which helps to analyze the user's gait characteristics; obtaining the plantar touching area and touching time by counting pressure areas and durations where the pressure exceeds a preset grounding pressure threshold, the plantar touching area and touching time being used to analyze the distribution of areas where the plantar foot touches the ground and the gait rhythm.

Finally, the first preset algorithm also extracts gait characteristics through the plantar pressure distribution map and plantar pressure change trend, the gait characteristics including stride length, stride speed, plantar landing mode and gait symmetry.

Specifically, stride length refers to the horizontal distance between two consecutive landings of the same side of the foot, reflecting the rhythmicity of the gait and the efficiency of the movement, and is obtained by recording the difference in the horizontal coordinates of the center of pressure of two consecutive touchdowns of the same side of the foot (e.g., the right foot), and stride speed refers to the number of strides accomplished per unit of time, directly reflecting the speed of the walking, and is obtained by recording the time interval between two consecutive touchdown events, and is obtained by analyzing the user's By analyzing the user's plantar pressure change cycle, the stride length and stride speed of each step can be calculated;

The plantar landing mode describes the order and pattern of different areas of the plantar foot contacting the ground during the gait cycle, and determines the plantar landing mode through the sequence of pressure value changes in the key pressure areas of the plantar foot, including heel landing, forefoot landing, or full-foot landing. For example, a heel pressure increase followed by a front metatarsal pressure increase would be a rearfoot landing, and a front metatarsal pressure increase followed by a heel pressure increase would be a front metatarsal landing, and different landing patterns may correspond to different gait problems or health conditions;

Gait symmetry is used to analyze the pressure distribution and gait rhythm of the left and right feet, to determine whether the user's gait is symmetrical or not, and to assess the overall symmetry by quantitatively calculating the difference between the key gait parameters of the left and right feet through the combination of the left and right foot stride lengths, touchdown time and other indexes in the gait cycle, high symmetry indicates good gait stability and coordination of movement, while low symmetry usually predicts a health problem, such as imbalance in the strength of the lower limbs or injury to the feet.

In one embodiment, the algorithm unit comprises a second preset algorithm for recognizing an abnormal pressure state, the second preset algorithm being a machine learning algorithm. The abnormal pressure state includes center of gravity shift, abnormal pressure distribution, gait instability, excessive internal and external rotation, or gait asymmetry, etc., and the second preset algorithm identifies the plantar pressure information exceeding the abnormal pressure threshold as an abnormal pressure state, and when the system detects an abnormal gait state of a user, it will alert the user through a feedback mechanism to remind the user to adjust the posture or activity mode in a timely manner, so as to prevent a long-term poor gait state from to prevent long-term poor gait from causing plantar health problems or further injuries.

Specifically, center of gravity shift refers to the fact that the center of gravity of the human body is not evenly distributed in the support area of the soles of the feet, which may lead to a decrease in the stability of the body and increase the risk of injury. The system can analyze the trajectory of the center of pressure movement of the user during walking, and the trajectory should be evenly distributed near the mid-axis of the plantar foot from the heel (initial contact) to the front metatarsal (push-off phase), and the average distance of the center of pressure trajectory deviating from the mid-axis of the plantar foot and the change of the angle with the ideal mid-axis of the plantar foot are calculated by the gait indicators for detection, and if an abnormal shift of the center of gravity in the gait is detected, and the distance and the angle of the angle exceed the abnormal pressure threshold value , then a center of gravity shift was determined, which could be due to a foot force imbalance or gait instability;

Abnormal pressure distribution includes too much or too little pressure, the plantar pressure should change dynamically during the gait cycle and show a reasonable distribution, if the pressure in some areas is too much, it may mean that the user has gait abnormality or bad posture, and it may indicate a foot injury or plantar structural problem, such as plantar fasciitis or metatarsal fracture, if the pressure in some areas is too little or not at all, especially after a long period of time of activity, it may also mean that the user has an incorrect gait, posture or underlying plantar health problem such as a collapsed arch or unstable gait. Abnormal pressure distribution is recognized when an area is subjected to sustained high pressure above the abnormal pressure threshold (commonly associated with poor gait or forcefulness) or when an area is subjected to transient pressure above the abnormal pressure threshold (commonly associated with strenuous exercise or accidental force);

Gait instability is a condition in which the user exhibits poor balance, abnormal gait, or unstable posture while walking. With the described gait indicators and gait characteristics, when the offset value exceeds the abnormal pressure threshold, the system can detect and recognize the unstable rhythm of gait or abnormal change in stride length, so as to identify whether the user has a problem of gait instability, which is particularly important in rehabilitation training or health monitoring of the elderly;

Excessive internal rotation means that the user walks with excessive pressure on the inside of the foot (big toe and arch) and low pressure on the outside of the plantar surface, which is usually associated with a collapsed arch (also known as flat feet) or muscle imbalance. Excessive external rotation means that the user walks with excessive pressure on the outside of the foot (pinky toe and outside of the heel) and insufficient pressure on the inside of the plantar surface, which is often seen in patients with high arched feet or unstable ankle joints. The dynamic trend of internal and external rotation can be assessed by comparing the ratio of pressure distribution on the inner and outer sides of the plantar foot to determine whether it is balanced or not, or by analyzing the bias of the center of gravity transfer trajectory in the gait indicators, and the abnormal pressure state is recognized when the abnormal pressure threshold is exceeded. If excessive internal and external rotation is detected, it may imply that the user has an incorrect posture or asymmetric weight bearing;

Gait asymmetry refers to the situation where the key parameters such as step length, step speed, touchdown time, pressure distribution and other key parameters of the left and right feet are significantly different during the user's walking process, which can be identified as an abnormal pressure state by comparing the plantar pressure information of the plantar regions of the left and right feet, with the degree of deviation exceeding the abnormal pressure threshold. The gait asymmetry may trigger chronic arthritis, imbalance of muscle strength and cause compensatory pain, etc.

Wherein, the abnormal pressure threshold can be custom set and adaptively and dynamically adjusted based on the user's historical data and the plantar pressure information to meet the individualized needs of different user groups, learn the user's daily activity patterns with the growth of the use time, and adaptively adjust for different user needs. The normal pressure range for athletes is significantly different from the normal pressure range for the elderly, and the system dynamically adjusts the threshold value by customizing the setting and learning the user's activity patterns and pressure data during subsequent use to ensure that the abnormal pressure detection is more personalized and accurate.

For example, for athletes, the system initially raises the pressure threshold to accommodate greater plantar loads, and when athletes train at high intensities, the system also raises the pressure threshold accordingly to avoid excessive feedback interference due to frequent exercise; for the elderly, the system initially sets a lower threshold for earlier detection of plantar health problems; and for rehabilitation patients, the system initially lowers the threshold to ensure that any slightest pressure is not detected. For rehabilitation patients, the system will initially lower the threshold to ensure that any slight abnormal pressure changes can be detected and fed back in time. Especially in the early stage of rehabilitation, the patient's plantar pressure distribution may be unstable, and the system will automatically increase the sensitivity of the feedback to help the patient adjust his/her posture in time; with the gradual change of the rehabilitation patient's gait and the plantar health condition, the system is able to track the patient's progress of rehabilitation automatically, and gradually increase the pressure threshold and adjust the parameters of pressure monitoring and feedback, allowing the patient to adjust the pressure and feedback parameters. monitoring and feedback parameters, so that patients can gradually adapt to the normal gait and activity intensity, to help patients recover faster and safer; and for users who gradually appear abnormal pressure state, the system is able to provide users with early warning through trend analysis, to help users take early action to prevent further deterioration of health problems; when the user suddenly accelerates or changes the gait, the system will quickly adjust the pressure data of the analysis model to avoid false alarms triggered by drastic changes in gait.

In one embodiment, the algorithm unit further comprises a third preset algorithm, through which a personalized plantar pressure health report is generated based on the user's historical data, the results of the first preset algorithm, and the results of the second preset algorithm, and the plantar pressure health report is transmitted to an external device through the wireless communication module, the plantar pressure health report including the abnormal pressure state, the plantar pressure distribution map and plantar pressure change trend, the gait indicators and gait characteristics, personalized health advice, and a long-term health trend analysis report.

Specifically, the personalized health advice gives rehabilitation training advice, gait adjustment advice, or health warning through in-depth analysis of the abnormal pressure state detection results. This function is particularly suitable for patients, athletes or the elderly who need long-term monitoring. For users who have been using the system for a long period of time, the system can also track changes in the health status and generate a long-term health trend analysis report to help the user detect gait abnormalities or foot problems at an early stage.

In a rehabilitation training scenario, the system can monitor the gait changes of rehabilitation patients in real time, determine the progress of rehabilitation and adjust the training program according to the actual situation; in a sports monitoring scenario, the system can be used for gait optimization of athletes, providing personalized training guidance for athletes by analyzing gait characteristics to reduce the risk of sports injuries.

In one embodiment, the algorithm unit further comprises a fourth preset algorithm for generating adjustment instructions and transmitting them to the adjustment module.

Specifically, when the plantar pressure measurement system is activated, the data processing module will perform an air pressure calibration to ensure that the initial air pressures of all airbags are within a standard range, and by analyzing the plantar pressure information to see if it is within a preset standard range, when the air pressure of certain the airbags is lower or higher than a standard value, generating an adjustment instruction to cause the adjustment module to inflate or deflate the air pressure within the airbags, to Ensuring that the airbags are in a proper working state, avoiding data errors caused by uneven air pressure in the airbags; during the operation of the plantar pressure measurement system, i.e., when the user is walking or exercising, the data processing module continuously monitors the air pressure state of each airbag and generates an adjustment instruction according to the real-time pressure change trend, so that the pressure in the airbags can be dynamically adjusted, and when it detects that the airbags are affected by the air pressure due to prolonged use or different gait pressures, the adjustment instruction is generated. When detecting a large fluctuation in the air pressure of the airbag due to prolonged use or the influence of the pressure of different gaits, the system will automatically make adjustments to keep the air pressure inside the airbag stable, ensuring that the system always maintains the optimal state of pressure monitoring, especially when the user uses it for a prolonged period of time or when switching between different gaits.

In one embodiment, the first preset algorithm, second preset algorithm, third preset algorithm and fourth preset algorithm are all capable of adaptively adjusting the parameters by the user, thereby adapting to the usage habits and gait characteristics of different users, which makes the system applicable not only to healthy people, but also to users with special needs for gait such as rehabilitation patients and the elderly.

Further, the system is capable of recognizing differences in pressure distribution under different activity states of the user. For example, the pressure distribution of the sole of the foot is different under different states such as standing, walking, running, etc. The system can adaptively adjust the parameters in the algorithm unit according to the different activity states to ensure that accurate analysis can be realized under various states.

The wireless communication module is connected to an external device or a cloud by means of a wireless communication protocol of Bluetooth or Wi-Fi to ensure stability and real-time data transmission, to facilitate remote monitoring by a user by transmitting real-time information of the plantar pressure and a health report of the plantar pressure to the external device or cloud, to facilitate remote monitoring by a user by transmitting user feedback inputted by a user from the external device or cloud to the data processing module, realizing personalized adjustment of parameters in the algorithm unit by the user;

Specifically, the user can view real-time information such as the plantar pressure distribution map and plantar pressure change trend, gait indicators, gait characteristics, and abnormal pressure state through the external device or cloud, and the user can review the user's historical data for comparative analysis at any time. Through the external device or cloud, the user is also able to further analyze the data and generate detailed health recommendations or training programs, for example, a rehabilitation therapist can adjust a patient's training program based on the plantar pressure health report, and an athlete can optimize gait and sports performance based on the plantar pressure health report.

Wherein the external device includes other smart devices such as a smartphone, a tablet computer, and the like. The wireless connection not only enables real-time transmission of data, but also facilitates data synchronization with a health management platform or a rehabilitation monitoring system, sharing the data with medical personnel or a rehabilitation trainer, and realizing remote health monitoring and rehabilitation guidance, and the medical personnel can also monitor the changes in the plantar pressure of the patient in real time, adjust the rehabilitation plan in time, and ensure that the patient is trained within a safe range.

The real-time feedback module is used to provide abnormal pressure state feedback to the user when an abnormal pressure state is detected, and the abnormal pressure state feedback includes vibration, sound, or visual feedback, and this instant feedback mechanism can effectively prevent potential injuries to the soles of the feet, especially in the process of exercise, when the user's gait or posture deviates from the normal range, the system can intervene in a timely manner, avoiding the prolonged bad posture on the plantar health damage;

Further, the user may customize on the external device or be assisted by a professional in setting and adjusting the mode, intensity, sensitivity and frequency of the abnormal pressure state feedback, and transmit the same to the real-time feedback module through the wireless communication module, and the real-time feedback module makes adjustments to meet the user's personal preferences or scene demands.

Wherein, with respect to the feedback mode, the sound feedback is suitable for receiving voice guidance in a quiet environment, and when selecting the sound feedback, the user can adjust the intensity of the feedback according to the degree of environmental noise; the visual feedback is suitable for occasions where the hearing is not sensitive or the vibration feedback is not obvious, for example, by means of the flash light of the cellular phone or the screen prompts to remind the user.

As far as the intensity of feedback is concerned, it can be customized and set according to the severity of the stress abnormality or the activity scenario, e.g. when severe stress is detected, the intensity of feedback will be increased to draw the user's attention. Vibration feedback is suitable for dynamic scenarios, e.g., the user selects a stronger vibration feedback during exercise and a lighter vibration alert during daily walking.

In terms of feedback sensitivity and frequency, a higher sensitivity may be set during rehabilitation training so as to receive alerts when a slight pressure abnormality occurs, during daily activities, the user may wish to reduce the feedback sensitivity and frequency to receive alerts only when a severe pressure abnormality occurs, and during high-intensity training, the athlete may wish to set a lower sensitivity in order to avoid frequent alerts.

Specifically, the vibration feedback is realized through a vibration motor embedded in the shoe, and the system triggers vibration when abnormal pressure state is detected, reminding the user to immediately adjust the gait or posture; the sound feedback is realized through an external device, and when abnormal pressure is detected, the system sends a signal to the external device through a wireless connection, and the external device emits sound feedback, reminding the user of the pressure abnormality; and the visual prompts are realized through The visual cues are realized through external devices, such as flashing lights or screen alarms, to remind the user to make adjustments.

The power management module adopts a low-power consumption design and includes a battery pack, a power monitoring circuit and an intelligent power controller, the battery pack being used to provide electric power, the power monitoring circuit being used to monitor the power status of the system in real time, and the intelligent power controller being used to control the power consumption of the plantar pressure detecting system to prolong the endurance time of the plantar pressure detecting system;

Specifically, by means of the power quantity monitoring circuit, when the battery power is lower than a set threshold, i.e., when the power quantity is insufficient, the system will give a low-battery warning in advance, prompting the user to recharge the battery to ensure the continuity of system operation;

automatically adjusting the power consumption according to the working state of the system through the intelligent power controller, the working state of the system includes a standby state (when the user does not act for a long period of time) and an action state (when the user starts walking or exercising), the power management module enters into a low-power mode under the standby state of the system, and the adjustment module, data processing module, wireless communication module, and real-time feedback module enter into a preset low-power mode to reduce energy consumption, wherein the air pressure sensor will reduce the acquisition frequency and reduce power consumption to ensure that the system can run continuously for a long time; the power management module resumes the normal working mode in the action state of the system, and the system will resume high-frequency acquisition and processing to ensure the real-time nature of gait analysis. The low-power consumption design ensures that the present application has low energy consumption characteristics when working for a long period of time in order to extend the battery life, thereby extending the duration of the plantar pressure measurement system and adapting to the user's continuous monitoring needs.

The second aspect of the present application proposes a method for detecting plantar pressure based on air pressure sensors and airbags, a flow block diagram of the method is shown in FIG. 2, which specifically comprises:

sensing the plantar pressure, obtaining the pressure data and transmitting the same synchronously to the data processing module, and adjusting the air pressure inside each of the airbags in accordance with the adjustment instruction through an adjustment module, the adjustment module comprises a plurality of independently working airbags, a plurality of air pressure sensors, an air pump and a plurality of air valves;

receiving, pre-processing the pressure data to obtain plantar pressure information, generating a plantar pressure health report and the adjustment instruction through a data processing module, the data processing module is connected to the air pressure sensor and comprises a pre-processing unit and an algorithm unit;

transmitting real-time the plantar pressure information and the plantar pressure health report to an external device or a cloud, and transmitting user feedback inputted by a user from the external device or the cloud to the data processing module through a wireless communication module;

providing abnormal pressure state feedback to the user when an abnormal pressure state is detected through a real-time feedback module, the abnormal pressure state feedback being provided in a manner comprising vibration, sound or visual feedback;

providing power, real-time monitoring the power status of the system, and controlling the power consumption of the plantar pressure measurement system through a power management module.

Wherein each of the airbags corresponds to a key pressure region embedded in the sole, the key pressure region corresponding to a key pressure point on the sole of the foot, comprising a heel region, a front metatarsal region, a lateral foot region, and a metatarsal arch region.

The foregoing is only a preferred embodiment of the present application, and is not intended to limit the present application, and the person skilled in the art should be able to realize that many examples can exist according to the basic method principles provided in the present application in combination with the actual situation, which should all be within the scope of protection of the present application without paying sufficient creative labor.

In the description of the present specification, reference is made to the terms “an embodiment”, “some embodiments”, “example”, “specific example”, or “a specific example”. ”, “some examples”, “exemplary”, “specific examples”, or “some examples”, etc. are described to mean that the specific features, structures, materials, or characteristics described in conjunction with the embodiment or example are included in at least one embodiment or example of the present application. In this specification, schematic expressions of the above terms are not necessarily directed to the same embodiment or example. Moreover, the specific features, structures, materials, or characteristics described may be combined in a suitable manner in any one or more embodiments or examples. Moreover, without contradicting each other, those skilled in the art may combine and combine different embodiments or examples and features of different embodiments or examples described herein.

It is also noted that in this specification, relational terms such as first and second are used only to distinguish one entity or operation from another, and do not necessarily require or imply the existence of any such actual relationship or order between these entities or operations. Furthermore, the terms “including”, “comprising”, or any other variant thereof, are intended to cover non-exclusive inclusion, such that a process, method, article, or apparatus comprising a set of elements includes not only those elements, but also other elements not expressly listed, or other elements not expressly listed for the purpose of such a process, method, article, or apparatus. elements, or which are inherent to such process, method, article or equipment.

What is claimed is:

1. A plantar pressure measurement system based on air pressure sensor and airbag, the system is embedded in the sole of a shoe, comprising an adjustment module, a data processing module, a wireless communication module, a real-time feedback module and a power management module;
the adjustment module comprises a plurality of independently working airbags, a plurality of air pressure sensors, an air pump and a plurality of air valves for sensing the plantar pressure, obtaining the pressure data and transmitting the same synchronously to the data processing module, and adjusting the air pressure inside each of the airbags in accordance with an adjustment instruction;
the data processing module is connected to the air pressure sensor and comprises a pre-processing unit and an algorithm unit for receiving, pre-processing the pressure data to obtain plantar pressure information, generating a plantar pressure health report and the adjustment instruction;
the wireless communication module for transmitting real-time the plantar pressure information and the plantar pressure health report to an external device or a cloud, and transmitting user feedback inputted by a user from the external device or the cloud to the data processing module;
the real-time feedback module for providing abnormal pressure state feedback to the user when an abnormal pressure state is detected, the abnormal pressure state feedback being provided in a manner comprising vibration, sound or visual feedback;
the power management module for providing power, real-time monitoring the power status of the system, and controlling the power consumption of the plantar pressure measurement system.

2. The plantar pressure measurement system of claim 1, wherein each of the airbags corresponds to a key pressure region embedded in the sole, the key pressure region corresponding to a key pressure point on the sole of the foot, comprising a heel region, a front metatarsal region, a lateral foot region, and a metatarsal arch region.

3. The plantar pressure measurement system of claim 1, wherein each of the airbags corresponds to one or more of the air pressure sensors; the adjustment module further comprises a weigh sensor; each time the plantar pressure information, plantar pressure health report, and the adjustment instruction obtained by the processing of the data processing module are stored in the system's memory or in the cloud as a user history data.

4. The plantar pressure measurement system of claim 1, wherein the algorithm unit comprises a first preset algorithm for generating a plantar pressure distribution map and a plantar pressure change trend, calculating a plurality of key gait indicators, and extracting a plurality of gait characteristics; the gait indicators comprise a center of pressure, a center of gravity transfer trajectory, as well as a plantar touching area and touching time; the gait characteristics comprise a step speed, a step speed, a plantar landing mode, and a gait symmetry.

5. The plantar pressure measurement system of claim 4, wherein the algorithm unit further comprises a second preset algorithm for identifying an abnormal pressure state;
the second preset algorithm is a machine learning algorithm;
the abnormal pressure state comprises center of gravity shift, abnormal pressure distribution, gait instability, excessive internal and external rotation, or gait asymmetry, and the second preset algorithm recognizes the plantar pressure information exceeding the abnormal pressure threshold as the abnormal pressure state;
the abnormal pressure threshold is adaptively and dynamically adjusted based on the user history data and the plantar pressure information.

6. The plantar pressure measurement system of claim 5, wherein the algorithm unit further comprises a third preset algorithm by which a personalized plantar pressure health report is generated based on the user history data, the results of the first preset algorithm, and the results of the second preset algorithm, and the plantar pressure health report is transmitted to the external device through the wireless communication module;
the plantar pressure health report comprises the abnormal pressure state, the plantar pressure distribution map and plantar pressure change trend, the gait indicators and gait characteristics, a personalized health advice, and a long-term health trend analysis report.

7. The plantar pressure measurement system of claim 1, wherein the algorithm unit further comprises a fourth preset algorithm for generating the adjustment instruction and transmitting it to the adjustment module for adjusting the air pressure of the airbag based on the adjustment instruction through the air pump and the plurality of air valves.

8. The plantar pressure measurement system of claim 1, wherein the power management module adopts a low power consumption design and comprises a battery pack, a power monitoring circuit and an intelligent power controller.

9. A plantar pressure measurement method based on air pressure sensor and airbag,
sensing the plantar pressure, obtaining the pressure data and transmitting the same synchronously to the data processing module, and adjusting the air pressure inside each of the airbags in accordance with the adjustment instruction through an adjustment module, the adjustment module comprises a plurality of independently working airbags, a plurality of air pressure sensors, an air pump and a plurality of air valves;
receiving, pre-processing the pressure data to obtain plantar pressure information, generating a plantar pressure health report and the adjustment instruction through a data processing module, the data processing module is connected to the air pressure sensor and comprises a pre-processing unit and an algorithm unit;
transmitting real-time the plantar pressure information and the plantar pressure health report to an external device or a cloud, and transmitting user feedback inputted by a user from the external device or the cloud to the data processing module through a wireless communication module;
providing abnormal pressure state feedback to the user when an abnormal pressure state is detected through a real-time feedback module, the abnormal pressure state feedback being provided in a manner comprising vibration, sound or visual feedback;
providing power, real-time monitoring the power status of the system, and controlling the power consumption of the plantar pressure measurement system through a power management module.

10. The plantar pressure measurement method of claim 9, wherein each of the airbags corresponds to a key pressure region embedded in the sole, the key pressure region corresponding to a key pressure point on the sole of the foot, comprising a heel region, a front metatarsal region, a lateral foot region, and a metatarsal arch region.
